(19) United States
(12) Patent Application Publication
(10) Pub. No.: US 2003/0186216 A1
(43) Pub. Date: Oct. 2, 2003

(54) Reaction measurement method for reactions using semiconductor nanoparticles, and quality evaluation method for semiconductor nanoparticles using the same

(75) Inventors: Keiichi Sato, Tokyo (JP); Susumu Kuwabata, Osaka (JP)

Correspondence Address: Reed Smith, Hazel & Thomas LLP, Suite 1400, 3110 Fairview Park Drive, Falls Church, VA, 22042-4503, US

(73) Assignee: Hitachi Software Engineering Co., Ltd.

(21) Appl. No.: 10/368,477

(22) Filed: Feb. 20, 2003

(30) Foreign Application Priority Data
Mar. 27, 2002 (JP) 2002-087768

(51) Int. Cl. C12Q 1/00; G01J 3/30; G01N 21/00; H01L 21/00; H01L 21/66

(52) U.S. Cl. 435/4; 356/317; 356/341; 438/1; 438/16

(57) ABSTRACT

Conventionally, reaction measurement for a reaction using semiconductor nanoparticles has been conducted by measuring absorbance and fluorescence intensity in a solution, and thus only a relative evaluation has been available, and its operation is complicated. The present invention provides an industrial evaluation method that is simply performed by conducting the measurement after a semiconductor nanoparticle solution is dried. Further, the measurement in a dry condition allows semiconductor nanoparticles to be applied to biopolymer microarrays or the like.

### BACKGROUND OF THE INVENTION

1. Technical Field

The present invention relates to a reaction measurement method for reactions using semiconductor nanoparticles that analyzes and evaluates emission intensity, emission characteristics or the like of the semiconductor nanoparticles, and a quality evaluation method for semiconductor nanoparticles using the above method.

2. Background Art

Semiconductor nanoparticles of a grain size of 10 nm or less are located in the transition region between bulk semiconductor crystals and molecules. Their physicochemical properties are therefore different from both bulk semiconductor crystals and molecules. In this region, the energy gap of a semiconductor nanoparticle increases as its grain size decreases, due to the appearance of a quantum-size effect. In addition, the degeneracy of the energy band that is observed in bulk semiconductors is removed and the orbits are dispersed. As a result, a lower-end of the conduction band is shifted to the negative side and an upper-end of the valence band is shifted to the positive side.

Semiconductor nanoparticles can be easily prepared by dissolving equimolar amounts of precursors of Cd and X (X being S, Se or Te). This is also true for manufacturing CdSe, ZnS, ZnSe, HgS, HgSe, PbS, or PbSe, for example. However, the semiconductor nanoparticles obtained by the above method exhibit a wide grain-size distribution and therefore cannot provide the full advantage of the properties of semiconductor nanoparticles. Therefore, attempts have been made to attain a monodisperse distribution by using chemical techniques to precisely separate the semiconductor nanoparticles having a wide grain-size distribution immediately after preparation into individual grain sizes and extract only those semiconductor nanoparticles of a particular grain size. The attempts that have been reported so far include an electrophoresis separation method that utilizes variation in the surface charge of a nanoparticle depending on the grain size, an exclusion chromatography that takes advantage of differences in retention time due to difference in grain size, and a size-selective precipitation method utilizing differences in dispersibility into an organic solvent due to difference in grain size. As a method that completely differs from the above methods, a size-selective optical etching method or the like has been reported wherein the grain size of semiconductor nanoparticles is controlled by irradiating a solution of semiconductor nanoparticles with monochromatic light.

Semiconductor nanoparticles obtained by these methods exhibit a spectrum with a relatively narrow wavelength-width peak. The obtained semiconductor nanoparticles are generally evaluated using the width narrowness and overall intensity of the spectrum as evaluation criteria, with the results for the evaluation criteria varying for each production lot. Measurement using absorbance is also conducted to investigate the uniformity of the grain size.

However, known processes for quality evaluation of semiconductor nanoparticles are conducted in solution by detecting light-emission or absorbance. When semiconductor nanoparticles in a solution are irradiated with excitation light, the intensity of excitation light and the intensity of fluorescence emission generated thereby are not linearly proportional but are dependent on the concentration of semiconductor nanoparticles. This is because, when the concentration of semiconductor nanoparticles is high, all the semiconductor nanoparticles are not equally irradiated with excitation light, or because the emitted fluorescence is shielded by other semiconductor nanoparticles. Likewise, the absorbance is also dependent on the concentration of semiconductor nanoparticles. Therefore, the quality evaluation method for semiconductor nanoparticles in a solution has poor operability, insufficient reliability in terms of the obtained emission amount, emission characteristics, absorbance, and absorption characteristics, and inferior reproducibility.

Hence, there is a need for a novel quality evaluation method having excellent operability, reliability, and reproducibility, for use instead of the conventional quality evaluation methods for semiconductor nanoparticles in solution.

Further, in various reactions using conventional semiconductor nanoparticles, optical characteristics or optical intensity such as fluorescence intensity or absorbance of semiconductor nanoparticles in solution have been obtained in order to measure the progress status of the reactions.

However, for the same reason as described above, the measurement methods for semiconductor nanoparticles in a solution have poor operability, insufficient reliability in terms of the emission amount, emission characteristics, absorbance, and absorption characteristics obtained from the reactant, and inferior reproducibility.

Thus, in order to measure the progress status of various reactions using conventional semiconductor nanoparticles, there is a need for a novel measurement method having excellent operability, reliability, and reproducibility, for use instead of optical methods for measurement in solution.

### SUMMARY OF THE INVENTION

The present inventors have found that the above problems are solved by measuring the optical intensity, optical characteristics and the like of semiconductor nanoparticles in a dry condition.

Namely, the present invention provides a reaction measurement method for a reaction using semiconductor nanoparticles, which comprises the steps of:

dropping a given volume of a semiconductor nanoparticle solution onto a substrate during and/or after the reaction using semiconductor nanoparticles;

drying the semiconductor nanoparticle solution on the substrate;

optically processing a dried product of semiconductor nanoparticles; and

conducting a reaction measurement for the reaction using semiconductor nanoparticles on the basis of the optical data.

Specific examples of the optical processing employed for the dried product of semiconductor nanoparticles include fluorescence emission measurement and absorbance measurement. Further, a method for processing the measurement results is not limited, and examples thereof include image processing using a fluorescence microscope or the like and computer analyses, and an exposure method using sensitive film.

Firstly, the above problems are solved when the optical processing of the dried product of semiconductor nanoparticles comprises irradiating the dried product of semiconductor nanoparticles with excitation light and obtaining the intensity of fluorescence emitted due to the irradiation.

Here, a total emission amount generated by the irradiation is obtained by computing the total sum of the intensity of the emitted fluorescence, and an emission amount per unit quantity can then be obtained.

Further, the dried product of semiconductor nanoparticles is irradiated with excitation light, fluorescence emitted due to the irradiation is captured as images using a scanner, and the captured images are processed and analyzed to obtain the emission intensity generated by the irradiation.

Secondly, the above problems are solved when the optical processing of the dried product of semiconductor nanoparticles comprises irradiating the dried product of semiconductor nanoparticles with light and obtaining the absorbance due to the irradiation.

In the present invention, the reactions using semiconductor nanoparticles are not particularly limited. Examples thereof include biopolymer detection reactions to detect the presence or absence of, and the amount of, binding to probe biopolymers, or vital observations carried out, for example, by: electrostatically binding a positively or negatively charged semiconductor nanoparticle to a negative or positive charge of a sample biopolymer; or inducing a reaction to amplify sample DNAs and RNAs, and performing a procedure such that semiconductor nanoparticles having their surface modified with a base or the like are incorporated therein during the reaction; or modifying a semiconductor nanoparticle surface with a functional group and modifying sample DNA through chemical binding therewith. Other examples of the reactions using semiconductor nanoparticles include stain reactions of living tissues in cell technologies.

Further, the present invention provides a method for quality evaluation of semiconductor nanoparticles, wherein the above method for measurement of reactions using semiconductor nanoparticles is employed.

Furthermore, the present invention provides a method for descriptive labeling of semiconductor nanoparticles, wherein the above method for measurement of reactions using semiconductor nanoparticles is employed.

As described above, measurement of reactions using semiconductor nanoparticles and quality evaluation of semiconductor nanoparticles have been conventionally conducted in solution, and reaction measurement and evaluation/analyses in a dry condition have not been carried out. In the present invention, attention is directed to the fact that semiconductor nanoparticles exhibit fluorescence characteristics such as fluorescence intensity and fluorescent spectrum, or absorption characteristics such as absorption intensity (abs.) or absorption spectrum even under a condition where a solution of the semiconductor nanoparticles is dropped onto a slide glass and dried. Furthermore, attention is also directed to the fact that, in a dry condition, optical measurement can be conducted without any transfer of electrons among individual semiconductor nanoparticles. This enables measurement and evaluation/analyses of reactions using semiconductor nanoparticles in a dry condition with greater accuracy than those carried out in solution.

The present invention is a reaction measurement method for reactions using semiconductor nanoparticles and for quality evaluation of semiconductor nanoparticles, without any limitation regarding a production method of the semiconductor nanoparticles. There are various methods for producing monodispersed semiconductor nanoparticles, and they are roughly classified into two types: one is a method to separate and extract semiconductor nanoparticles according to their grain size after their preparation; and the other is a method to prepare semiconductor nanoparticles that are already monodispersed. Examples of the former type include an electrophoresis separation method that utilizes the fact that the surface charge of a nanoparticle varies in accordance with grain size, exclusion chromatography that takes advantage of differences in retention time due to differences in grain size, and a size-selective precipitation method utilizing differences in dispersibility into an organic solvent due to differences in grain size. Examples of the latter type include a high temperature growth method that allows precursors to react with each other and grow under a high temperature environment to enable control of grain size by means of the reaction time, a size-selective optical etching method that controls the grain size by irradiating a semiconductor nanoparticle solution with a monochromatic light, and a reversed micelle method that controls grain size by means of the volume ratio between an organic solvent and an aqueous solvent utilizing a surfactant.

The materials for semiconductor nanoparticles are not limited, and preferable examples of the materials include ZnO, ZnS, ZnSe, ZnTe, CdO, CdS, CdSe, CdTe, HgS, HgSe, HgTe, InP, InAs, GaN, GaP, GaAs, TiO₂, WO₃, PbS, and PbSe.

Semiconductor nanoparticles prepared by the above methods have generally been evaluated by measuring absorbance and fluorescence intensity in a solution. A feature of the present invention is that measurement and evaluation/analysis of semiconductor nanoparticles is conducted in a dry condition, and not in a solution.

### BRIEF DESCRIPTION OF THE DRAWINGS

FIG. 1 is a conceptual view of a semiconductor nanoparticle solution dropped onto a substrate.

FIG. 2 illustrates an example display view of analysis by a computer.

FIG. 3 is a schematic view illustrating DNA detection using chemically modified semiconductor nanoparticles.

### DETAILED DESCRIPTION OF THE INVENTION

### EXAMPLE 1

An evaluation method and an analysis method for semiconductor nanoparticles will be herein described.

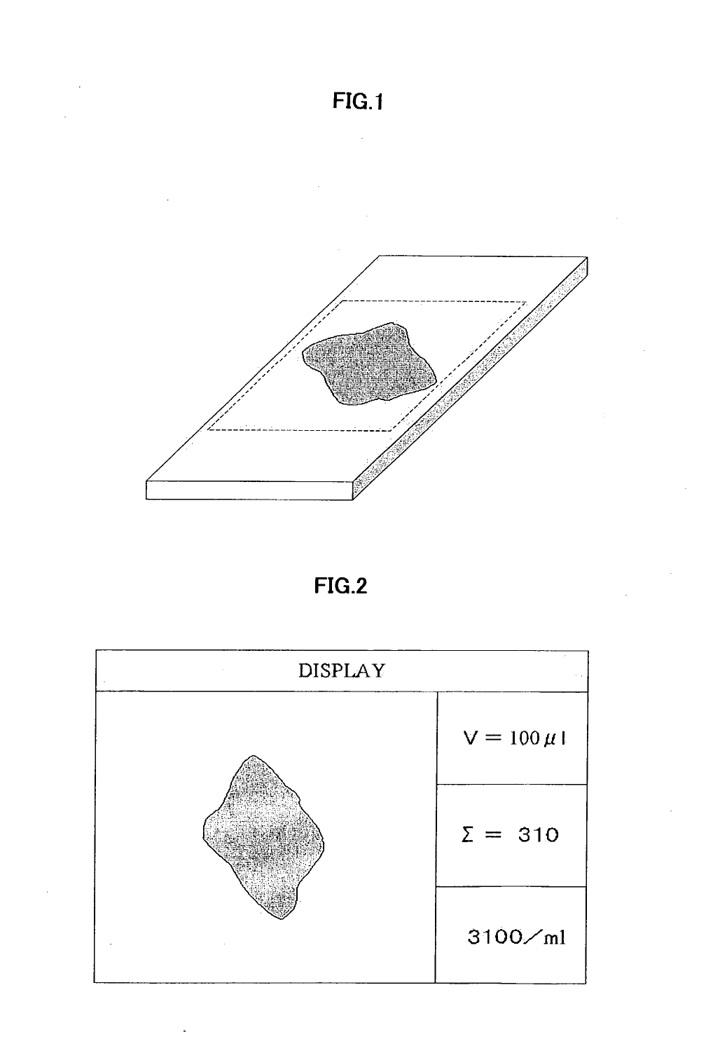

Semiconductor nanoparticles used herein for the measurement and evaluation were prepared as follows: 1000 ml of an aqueous solution of sodium hexametaphosphate (0.1 mmol) and cadmium perchlorate hexahydrate(0.2 mmol) was prepared and adjusted to pH 10.3, and hydrogen sulfide gas (0.2 mmol) was then injected into the solution while stirring vigorously. The obtained semiconductor nanoparticles were monodispersed by the size-selective photocorrosion. An aqueous solution of the monodispersed CdS semiconductor nanoparticles thus prepared was dropped onto a substrate such as a slide glass and dried (FIG. 1). Thereafter, the aqueous solution was irradiated with excitation light of a wavelength of approximately 350 nm.

The results were turned into images by a reading apparatus comprising a fluorescence microscope (OLYMPUS OPTICAL CO., LTD., BX60), the images were analyzed by a computer, and integration processing of a fluorescence region was carried out. As a result, the total light emission of semiconductor nanoparticles on the slide glass could be obtained, and further, the light emission per unit quantity could be obtained by correlating the total light emission with the volume of the dropped semiconductor nanoparticle solution (FIG. 2).

Alternatively, the evaluation can be conducted by irradiating a designated region with excitation light and determining an optical spectrum. In addition, calculating the correlation between absorbance and fluorescence amount and the correlation among a plurality of spectra enable more accurate evaluation.

### EXAMPLE 2

A method for measurement of reactions using semiconductor nanoparticles (biopolymer analysis method using semiconductor nanoparticles) will be herein described.

Conventionally, in the detection of biopolymers using semiconductor nanoparticles, evaluation has been conducted by measuring optical spectra mainly in solution. However, it has been extremely difficult to conduct analyses in solution by means of biopolymer microarrays as typified by DNA microarrays.

The present invention enables analyses with biopolymer microarrays by measuring the amount of semiconductor nanoparticles under a dry condition.

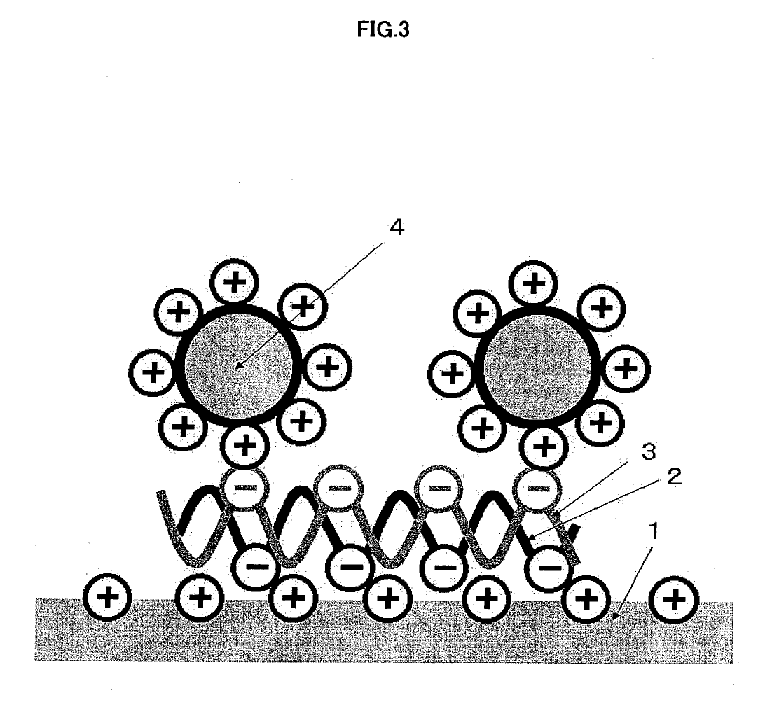

The present applicant filed an application (Japanese Patent Application No. 2002-27616) for an invention wherein a semiconductor nanoparticle having a chemically modified surface is used as a detection reagent for proteins or DNA. A mechanism for the detection of biopolymers using a semiconductor nanoparticle having a chemically modified surface will now be described referring to FIG. 3. In FIG. 3, by binding between a positive charge of a surface substrate 1 forming a planar shape or bead shape and a negative charge of a phosphate side-chain of a probe DNA 2, the probe DNA 2 was immobilized to the substrate 1. The probe DNA 2 and a sample DNA 3 then hybridized to each other through hydrogen bonding. As a result, a negative charge of a phosphate side-chain of the sample DNA 3 increased. A positively charged semiconductor nanoparticle 4 bound to the negative charge of the sample DNA 3, and based on the amount of bound semiconductor nanoparticles, information concerning the hybridized sample DNA 3 was provided as a signal. In the example of FIG. 3, the probe DNA 2 and the semiconductor nanoparticle 4 were negatively and positively charged, respectively, but the charges may be the reverse thereof. Proteins and the like have isoelectric points, and thus the charge of the sample DNA 3 varies between positive and negative depending on the fluctuation of pH values. When the sample DNA 3 is positively charged, a negatively charged semiconductor nanoparticle 4 may be used.

Modification of a semiconductor nanoparticle with a functional group can be easily carried out by admixing a thiol compound into a semiconductor nanoparticle solution to induce a substitution reaction between the semiconductor nanoparticle surface and the thiol compound. Herein, an analytical method with a DNA microarray using a semiconductor nanoparticle modified with a functional group will be exemplified. An application example wherein a reaction surface is modified with thiocholine ((2-mercaptoethyl)trimethylammonium) is described.

350 mg of acetyl thiocholine iodide was dissolved into 1.2 cm³ of nitrogen-saturated 2 mol·dm⁻³ HCl aqueous solution, and the mixture was allowed to stand for 12 hours at room temperature. 0.2 cm³ of 28% ammonia water in a nitrogen atmosphere was added to the mixture for neutralization, to thus prepare alkalescent 0.86 mol·dm⁻³ thiocholine ((2-mercaptoethyl)trimethylammonium) aqueous solution. By modifying the nanoparticle surface with this aqueous solution, thiocholine-modified CdS nanoparticles having a positive charge on the particle surface were prepared. 4.65 ml of the aqueous solution was added to a CdS nanoparticle solution after size-selective optical etching, and the resultant solution was left under stirring for 24 hours at room temperature. The thus obtained semiconductor nanoparticles were positively charged and easily adsorbed onto negatively charged DNAs or proteins or the like. Next, an example of application to a DNA microarray using this property will be described. A DNA microarray is used for a method wherein: a large number of known probe DNAs are chemically immobilized onto a substrate; sample DNAs to be assayed are then introduced on top of the probes; and the sequence characteristics of the samples are identified based on the presence or absence of, and the amount of, binding between the probe DNAs and sample DNAs. Until now, the following method has been generally used to determine the existence of DNA binding and the amount of binding. That is, samples are modified with fluorescent substances or radioactive substances, and the existence of binding and the amount thereof is then determined by optically detecting these substances. The present invention does not require pre-treatment for modifying samples, and thus has a feature of requiring no sample pre-treatment by RNA reverse transcription or PCR reaction.

A sample DNA solution was dropped on a DNA microarray, and a cover glass was gently placed on the mixture. Then, the mixture was reacted for 16 hours under a hermetically closed environment using CHBIO (Hitachi Software Engineering Co., Ltd.).

After the reaction, a slide glass was taken out therefrom, and the slide glass was soaked in a 2×SSC, 0.1% SDS solution and the cover glass was removed. Then, the slide glass was soaked for 2 hours in a 2×SSC, 0.1% SDS solution having a CdS semiconductor nanoparticle concentration of 1.2×10¹⁷ mol·dm⁻³. Thereafter, the slide glass was shaken in a 2×SSC, 0.1% SDS solution for 20 minutes at room temperature and then shaken in a 0.2×SSC, 0.1% SDS solution for 20 minutes at room temperature. Further, in order to remove non-specific adsorptive samples, the slide glass was shaken in 0.2×SSC, 0.1% SDS solution for 20 minutes at 55° C., and the same operation was repeated. Thereafter, the slide glass was shaken several times in 0.2×SSC, 0.1% SDS solution at room temperature, and then shaken several times at room temperature in solutions of 0.2×SSC and 0.05×SSC, respectively. The above soaking and washing steps were carried out using a staining jar. The slide glass was then centrifuged and dried, and provided for analysis which was performed by filtering only the fluorescence wavelength of semiconductor nanoparticles with an epi-illumination fluorescence microscope. As a result, bright red fluorescence was measured from each spot of the DNA microarray. According to the present example, it was found that the optical measurement of semiconductor nanoparticles in a dry condition was useful for reaction measurement for various reactions using semiconductor nanoparticles.

The present method is applicable to biopolymer microarrays in general. It can be applied not only to DNA microarrays but also to other biopolymer microarrays and sensors such as protein microarrays, which have the same principle.

### EFFECTS OF THE INVENTION

According to the present invention, it is possible to conduct reaction measurement for a reaction using semiconductor nanoparticles in a simple and efficiently reproducible manner. Further, quality evaluation of semiconductor nanoparticles can be carried out simply, enabling explicit description of quality indexes.

What is claimed is:

1. A reaction measurement method for a reaction using semiconductor nanoparticles, comprising the steps of:
dropping a given volume of a semiconductor nanoparticle solution on a substrate during and/or after the reaction using the semiconductor nanoparticles;
drying the semiconductor nanoparticle solution on the substrate;
optically processing a dried product of semiconductor nanoparticles; and
measuring the reaction using the semiconductor nanoparticles on the basis of the optical data..

2. The reaction measurement method according to claim 1, wherein the optical processing of the dried product of the semiconductor nanoparticles comprises irradiating the dried product of the semiconductor nanoparticles with excitation light and obtaining an intensity of fluorescence emitted due to the irradiation.

3. The reaction measurement method according to claim 2, wherein a total emission amount due to the irradiation is obtained by computing a total sum of the intensity of the emitted fluorescence, and an emission amount per unit quantity is further obtained.

4. The reaction measurement method according to claim 2 or 3, wherein the dried product of the semiconductor nanoparticles is irradiated with excitation light, fluorescence emitted due to the irradiation is captured as an image using a scanner, and the captured image is processed and analyzed to obtain the emission intensity generated by the irradiation.

5. The reaction measurement method according to claim 1, wherein the optical processing of the dried product of the semiconductor nanoparticles comprises irradiating the dried product of the semiconductor nanoparticles with light to obtain the absorbance generated by the irradiation.

6. The reaction measurement method according to any of claims 1 to 5, wherein the reaction using semiconductor nanoparticles is a biopolymer detection reaction to detect the presence or absence of, and the amount of, binding to a probe biopolymer by electrostatically binding a positively or negatively charged semiconductor nanoparticle to a negative or positive charge of a sample biopolymer.

7. The reaction measurement method according to any of claims 1 to 5, wherein the reaction using semiconductor nanoparticles is a stain reaction of a living tissue.

8. The reaction measurement method according to any of claims 1 to 5, wherein the reaction using semiconductor nanoparticles is a modification reaction of a biopolymer.

9. A quality evaluation method for semiconductor nanoparticles, wherein the method for measurement of a reaction using semiconductor nanoparticles of any of claims 1 to 5 is employed.

10. A method for descriptive labeling of a semiconductor nanoparticle, wherein the method for measurement of a reaction using semiconductor nanoparticles of any of claims 1 to 5 is employed.
